(19) United States
(12) Patent Application Publication
(10) Pub. No.: US 2007/0292479 A1
(43) Pub. Date: Dec. 20, 2007

(54) Film-shaped drug forms for use in the oral cavity (wafers)

(76) Inventors: Hans-Peter Podhaisky, Berlin (DE); Stefan Bracht, Glienicke Nordbahn (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/626,625

(22) Filed: Jan. 24, 2007

(30) Foreign Application Priority Data
Jan. 24, 2006 (DE) 10 2006 003 512.7

(51) Int. Cl. A61K 8/02; A61K 31/565; A61K 8/73; A61P 5/24; A61K 31/57

(52) U.S. Cl. 424/435; 424/488; 514/177; 514/178; 514/182

(57) ABSTRACT

The present invention describes film-shaped systems capable of transmucosal buccal application and comprising at least one steroid hormone from the group of the androgens, the progestogens and the oestrogens, 10-70% by weight of a cyclodextrin or a cyclodextrin derivative, and a film-forming agent disintegrating in an aqueous medium.

Film-shaped drug forms for use in the oral cavity, referred to below as wafers, offer a number of advantages compared to conventional drug forms. Through the direct absorption of the active substance through the oral mucosa, the gastrointestinal tract is bypassed, as a result of which an alternative presentation is demonstrated for drug substances which because of their metabolisation by the liver (“first pass metabolism”) can otherwise only be administered parenterally or transdermally. Further, the presentation is especially suitable for older patients with decreased saliva flow and swallowing difficulties associated therewith or also for small children.

The production of wafers is effected by mixing drug substances with water-soluble film-forming agents such as for example celluloses, starches, gelatines or polyacrylates, followed by film coating onto a dehesive support, drying, singulation and packing.

For the case where lipophilic drug substances (lipophilic drug substances in the sense of the present invention means drug substances with a partition coefficient between 1-octanol and water [Log P_{Oct/H2O}≧2) are to be incorporated into the wafers, the solubility in the water-soluble film-forming agents is often insufficient to dissolve the drug substances directly in the coating solution. In this case, the state of the art is to take the drug substances up in an organic solvent before the mixing with the aqueous film-forming agent or else to bring them into solution using emulsifiers. The procedure described can however have disadvantages. In case of the use of organic solvents, the dried films still have a residual content of organic solvents, which places special requirements on the validation of the production methods and the analytical method for the medicament. Thus the packing of Triamminic® by Novartis results in both acetone and also ethanol as an inactive component. For ethanol there is the note: less than 5%, which makes use by small children seem problematic, and rules out use by alcoholic patients.

A further disadvantage due to processing with solvents can arise if, after the drying of the film and the evaporation of the solvent, the solvent power of the water-soluble film-forming agent for the drug substance is exceeded. In this case, crystallisation of active substance in the polymer matrix can occur. The latter can have adverse effects on the transbuccal absorption rates and on the stability of the drug form.

A further option for incorporating a lipophilic drug substance into aqueous film-forming agents is the use of emulsifiers. Thus for example US 20040115137 inter alia proposes the use of Polysorbate 80 at a preferred concentration of 1-5%. A problem in the use of polysorbate 80 in this concentration range or of related substances as solubility mediators for lipophilic drug substances is their often soapy taste, which can be a disadvantage for the acceptance of a transbuccal application.

### STATEMENT OF PURPOSE

One purpose of the invention is to develop an aqueous coating solution for the production of a wafer which contains a lipophilic drug substance which is fully dissolved in the aqueous coating solution, and which moreover also remains fully dissolved in the dried film, so that the dried film appears essentially transparent (essentially transparent in the sense of the present invention means films which exhibit visible light transmission of ≧50%) and contains no visible residues of undissolved active substance.

A further purpose of the invention is to provide such a coating solution which avoids the said disadvantageous use of organic solvents or emulsifiers.

A further purpose of the invention is that the dried film has good mucoadhesive properties, that is, that after the placing of the wafer in the mouth and attachment to the mucosa of the palate, the film remains there until complete dissolution.

In addition, the wafer should display good taste properties, in particular even when an unpleasant-tasting drug substance has to be processed. Unsatisfactory taste properties are probably a major reason why wafers have so far barely become established on the market, as compared to conventional drugs.

A further purpose of the invention is to provide a wafer that disintegrates in a few minutes in an aqueous environment and is at least so flexible that under shear stress associated with its use (removal from the pack and attachment to the oral mucosa) there is no danger of tearing or splitting of the film.

### SOLUTION OF THE PROBLEM

The present problem is solved by a film-shaped system capable of transmucosal buccal application and comprising

at least one steroid hormone from the group of the androgens, the progestogens and the oestrogens,

10-70% of a cyclodextrin or a cyclodextrin derivative, and

a film-forming agent disintegrating in an aqueous medium.

According to the invention, the incorporation of lipophilic drug substances into the aqueous polymer solution of the wafer is effected through the use of complexing agents from the group of cyclodextrins such as for example beta-cyclodextrin and gamma-cyclodextrin and also cyclodextrin derivatives, in particular of hydrophilic descendants of beta-cyclodextrin such as HP-beta cyclodextrin or sulfobutyl ether-beta cyclodextrin sodium salt (Captisol®).

These inclusion compounds can then be mixed with the aqueous polymer solutions, coated as a film, dried and singulated, so that substantially transparent wafers are formed.

Water-soluble polymers from the group of polyvinyl alcohols with a degree of hydrolysis of 75-99% (e.g. Mowiol® grades from Kuraray Specialties Europe), polyvinylpyrrolidone, polyethylene glycols, hydrophilic cellulose derivatives such as hydroxypropylcellulose, pullulan or maltose, hydrophilic starch derivatives such as carboxymethyl starch, alginates or gelatines and other polymers known from the prior art are suitable for the preparation of the water-soluble films. Reference is made for this purpose to the documents DE 2432925, DE 19956486A1, DE 19652257A1, DE 19652188, DE 10107659 and WO 03/011259 A1, including the prior art cited therein. Preference is given to the use of polyacrylates and polyvinyl alcohols.

Partially hydrolyzed polyvinyl alcohols of medium viscosity such as for example Mowiol 40-88 and methacrylic acid copolymers such as for example Eudragit L100-55 are found to be especially suitable.

Surprisingly, the incorporation here of a high mass content of the cyclodextrin compound, relative to the mass content of the water-soluble polymer, has an advantageous effect on the film properties and the use properties of the wafers.

According to the invention, the incorporation of a 20-60 or even a 10 to 70% content of HP-beta cyclodextrins or Captisol (w/w, based on the dry weight of the wafer) is found to be advantageous and incorporation of a content of cyclo-dextrin ≧40% (w/w, based on the dry weight of the wafer) is found to be especially advantageous for the film and use properties of the wafer, in that the films obtained are less brittle and fragile and more tear-resistant compared to films composed of pure polymer.

The wafers produced from coating solutions according to the invention further have improved taste properties compared to those which consist of pure polymer. This is due to the slightly sweet taste of the cyclodextrins, or, in the case of the sulfobutyl ether-beta cyclodextrin sodium salt (Captisol®) the improvement in taste is a result of the slightly salty taste.

Through the reduction of the mass content of the swellable, water-soluble polymer in the replacement with cyclodextrin, the hygroscopicity of the preparation decreases and the dissolution of the wafer in water is encouraged without undesired swelling. During use in the mouth, the mouth feel of the films produced according to the invention therefore seems less slimy than that of pure polymer films.

It is therefore advantageous that the wafer can consist of up to 70% (based on dry weight) HP-beta cyclodextrin or Captisol.

In addition, the wafers according to the invention dissolve more quickly than those which consist of pure polymer films, since the dissolution-retarding effect of polymer swelling occurs to a lesser extent, if at all.

The superiority of a preparation according to the invention will be illustrated in more detail in the following example. For this, two wafers were compared, one of which (wafer B) had a composition according to the invention.

Two aqueous coating solutions of the following composition were produced in a manner known to the skilled person.

| Wafer A: Coating solution with | | Wafer B: Coating solution | |
|---|---|---|---|
| no addition of cyclodextrin | | according to the invention | |
| | Content [%] | | Content [%] |
| | v/v in | | v/v in |
| | aqueous | | aqueous |
| | coating | | coating |
| Component | solution | Component | solution |
| Drospirenone | 1 | Drospirenone | 1 |
| Polyvinyl alcohol | 20 | Polyvinyl alcohol | 10 |
| (Mowiol 40-88) | | (Mowiol 40-88) | |
| Water | ad 100 | HP-beta cyclodextrin | 10 |
| | | Water | ad 100 |

To this end, the cyclodextrin derivative was dissolved in water and then the polyvinyl alcohol (Mowiol) was sprinkled onto the solution and dissolved, if appropriate by heating to 70° and stirring, to form a clear solution.

The aqueous coating solution was coated onto a suitable dehesive support material such as for example polyethylene (PE) or polyethylene terephthalate (PET) films and dried initially for about 10 minutes at 60° C. and then for about 10 minutes at 105° C. in a drying cabinet.

The basis weight of the dried film was about 80 g/m². The dried film was singulated into 5×5 cm pieces, and the wafers were manually peeled off the dehesive support film.

Wafers with the following composition were obtained:

| | | Wafer B (produced from the coating | |
|---|---|---|---|
| Wafer A: | | solution according to the invention) | |
| | Content [%] | | Content [%] |
| | v/v in | | v/v in |
| Component | dried wafer | Component | dried wafer |
| Drospirenone | 4.5 | Drospirenone | 4.5 |
| Polyvinyl alcohol | 91 | Polyvinyl alcohol | 45.5 |
| (Mowiol 40-88) | | (Mowiol 40-88) | |
| Water | ad 100 | HP-beta cyclodextrin | 45.5 |
| | | Water | ad 100 |

Both wafers were then tested by a volunteer.

Wafer A adhered well to the palate, had a slimy (bitter taste), which was perceptible for 4-5 minutes, and had then so far dissolved that it was no longer perceptible.

Wafer B adhered well to the palate, the taste was largely neutral and appreciably less slimy than the mouth feel of wafer A. After 1-2 minutes, the preparation had so far dissolved that it was no longer perceptible.

A further advantage of the addition according to the invention of 10-70% HP-beta cyclodextrin or Captisol® to the aqueous coating solutions compared to cyclodextrin-free coating solutions can be achieved in the processing of drug substances which have an unpleasant, for example bitter, taste.

One proposal for covering an unpleasant taste of the active substance of a wafer is put forward by WO 03070227, which proposes the use of carbon dioxide-forming agents such as for example sodium hydrogen carbonate, sodium carbonate, potassium carbonate and potassium hydrogen carbonate in combination with an acid component such as for example citric acid, tartaric acid, adipic acid, malic acid and ascorbic acid. However, WO 03070227 does not indicate any suitable way whereby such a preparation can be produced. Since the carbon dioxide-forming agent releases carbon dioxide directly on contact with water, the incorporation of the carbon dioxide-forming agent into the aqueous coating matrix of the wafers would have to take place with exclusion of water, which seems almost impossible, since even the residual moisture of the dried films is sufficient to start the reaction.

A further approach to the masking of a bitter taste of a drug substance of a wafer is the addition of flavourings and sweeteners. Thus EP 1588701 describes a wafer for the transbuccal administration of nicotine. The application proposes the addition of various flavourings and sweeteners: “further, as flavourings, vanilla flavouring, orange flavouring, orange-cream flavouring, strawberry flavouring, raspberry flavouring or chocolate flavouring can be added, each alone or in combination. In addition, one or more sweeteners such as sucralose, aspartame, cyclamate, saccharine and acesulfam and salts thereof can be added.”

Such a procedure can however also have disadvantages: the intrinsic taste of the said flavourings and additives can be experienced as unpleasant, especially when addition of several percent of flavouring or sweetener (based on the dry weight of the wafer) is necessary to mask an extremely bitter drug substance.

It is therefore desirable to use an additive which obliterates or at least greatly reduces the unpleasant drug substance taste, without producing an excessive taste of its own.

With the addition according to the invention of HP-beta cyclodextrin or Captisol, a solution for the said problem can be indicated. Cyclodextrins are suitable for masking the undesired taste of orally administered drugs (Szejlti and Szente 2005, Eur J Pharm Biopharm). Both HP-beta cyclodextrin (slightly sweet) and also Captisol (slightly salty) have an unobtrusive intrinsic taste, so that a wafer according to the invention seems approximately neutral in taste.

The invention is more particularly elucidated in the following example:

An aqueous inclusion compound of the active substance drospirenone in HP-beta cyclodextrin is produced in a manner known to the skilled person. To this end, the required amount of drospirenone can be incorporated for example directly in a 35% mm aqueous solution of HP-beta cyclodextrin and made to form a clear solution by stirring.

Subsequently, the required amount of this presolution is mixed with an aqueous polyvinyl alcohol solution to form the final coating solution. In the case of Mowiol 40-88, for example, a 12% mm aqueous polymer solution obtained by dissolving the polymer in water, if appropriate by heating to 70° C. and stirring, can be used.

Alternatively, the cyclodextrin is initially dissolved in the total amount of water for the coating solution by stirring. The drospirenone is then dissolved in this solution by stirring. Finally, the polymer is added and by stirring and also if appropriate heating to 70° C. a clear coating solution is produced.

### EXAMPLE 1

| | Content [%] | |
|---|---|---|
| | v/v in | Content [%] |
| | aqueous coating | m/m in |
| Component | solution | dried wafer |
| Drospirenone | 1 | 5 |
| HP-beta cyclodextrin | 9.3 | 47 |
| Polyvinyl alcohol (Mowiol 40-88) | 9 | 45 |
| Water | ad 100 | ad 100 |

This aqueous solution is coated onto a suitable dehesive support material such as for example polyethylene (PE) or polyethylene terephthalate (PET) films, dried and individualized.

A visually transparent, slightly sticky, water-soluble film is formed which is characterised by approximately neutral taste.

In addition, a coating solution according to the invention can have incorporated into it a combination of at least two hormones, one selected for example from the group of the progestogens and another from the group of the oestrogens. After the production of the aqueous inclusion compound formed from HP-beta cyclodextrin/active substance, the further processing of the present invention's coating solution into wafers does not differ from the procedure shown under Example 1. Examples of such preparations according to the invention which comprise a combination of two hormones are set out hereinbelow:

### EXAMPLE 2

| | Content [%] | |
|---|---|---|
| | v/v in | Content [%] |
| | aqueous coating | m/m in |
| Component | solution | dried wafer |
| Drospirenone | 1 | 5 |
| Ethinyloestradiol | 0.015 | 0.075 |
| HP-beta cyclodextrin | 9.3 | 47 |
| Polyvinyl alcohol (Mowiol 40-88) | 9 | 45 |
| Water | ad 100 | ad 100 |

### EXAMPLE 3

| | Content [%] | |
|---|---|---|
| | v/v in | Content [%] |
| | aqueous coating | m/m in |
| Component | solution | dried wafer |
| Drospirenone | 1 | 4.9 |
| Oestradiol | 0.5 | 2.4 |
| HP-beta cyclodextrin | 9.3 | 45.2 |
| Polyvinyl alcohol (Mowiol 40-88) | 9 | 43.7 |
| Water | ad 100 | ad 100 |

By adding further excipients to the coating solutions of the present invention, selected physical parameters of the wafers produced using the respective coating solution can be controlled in a specific manner.

For instance, by the use of plasticizers such as for example 1,2 propylene glycol or polyethylene glycol, the tear resistance of the films can be optimized.

Through the addition of disintegration accelerators such as for example sodium carboxymethylcellulose (NA-CMC), Ludipress or Kollidon CL, accelerated disintegration of the films in the aqueous medium can be attained and furthermore the flavour and feel properties of the films can be modified. Thus for example films can be obtained which have a slightly grainy feel in the mouth.

By addition of flavourings such as for example sorbitol the taste properties and the residual moisture content of the films can be varied.

Through the addition of microcrystalline cellulose (MCC) the films acquire a more paper-like structure.

Further examples of aqueous coating solutions according to the present invention, each including one of the aforementioned additions, are indicated below:

### EXAMPLE 4

| | Content [%] | |
|---|---|---|
| | v/v in | Content [%] |
| | aqueous coating | m/m in |
| Component | solution | dried wafer |
| eF-MENT | 1 | 3.2 |
| Captisol | 10 | 32 |
| Polyacrylic acid | 10.7 | 34.3 |
| (Eudragit L100-55) | | |
| 1,2-Propylene glycol | 8 | 25.6 |
| Water | ad 100 | ad 100 |

### EXAMPLE 5

| | Content [%] | |
|---|---|---|
| | v/v in | Content [%] |
| | aqueous coating | m/m in |
| Component | solution | dried wafer |
| Ethinyloestradiol | 0.4 | 1.6 |
| Gestodene | 1 | 4 |
| HP-beta cyclodextrin | 10 | 40 |
| Polyvinyl alcohol (Mowiol 40-88) | 10.7 | 42.9 |
| Sodium carboxymethyl- | 2 | 8 |
| cellulose | | |
| Water | ad 100 | ad 100 |

### EXAMPLE 6

| | Content [%] | |
|---|---|---|
| | v/v in | Content [%] |
| | aqueous coating | m/m in |
| Component | solution | dried wafer |
| Ethinyloestradiol | 0.4 | 1.6 |
| Gestodene | 1 | 4 |
| HP-beta cyclodextrin | 8.8 | 35.8 |
| Polyvinyl alcohol (Mowiol 40-88) | 7.7 | 31.3 |
| Kollidon CL | 5.5 | 22.4 |
| Water | ad 100 | ad 100 |

### EXAMPLE 7

| | Content [%] | |
|---|---|---|
| | v/v in | Content [%] |
| | aqueous coating | m/m in |
| Component | solution | dried wafer |
| Testosterone | 1 | 4.6 |
| HP-beta cyclodextrin | 9.3 | 42.5 |
| Polyvinyl alcohol (Mowiol 40-88) | 9 | 41 |
| Sorbitol | 1 | 4.6 |
| Water | ad 100 | ad 100 |

### EXAMPLE 8

| | Content [%] | |
|---|---|---|
| | v/v in | Content [%] |
| | aqueous coating | m/m in |
| Component | solution | dried wafer |
| Testosterone | 1 | 4.3 |
| HP-beta cyclodextrin | 9.8 | 42.6 |
| Polyvinyl alcohol (Mowiol 40-88) | 9.4 | 41.0 |
| AvicelPH101 (MCC) | 2 | 8.7 |
| Water | ad 100 | ad 100 |

The compositions named in Examples 1-8 can be varied in so far as they contain as the pharmaceutically active component at least one hormone which is selected from the group which includes the androgens, for example testosterone, 7alpha-methyl-19-nortestosterone (MENT), MENT-17-acetate, 7alpha-methyl-11beta-fluoro-19-nortestosterone (eF-MENT), eF-MENT-17-acetate, testosterone propionate, testosterone undecanoate, testosterone oenanthate, mesterolone, nandrolone decanoate, clostebol acetate, metenolone acetate, the oestrogens, for example 17-beta-oestradiol, ethinyloestradiol, oestradiol valerate, oestradiol cypionate, oestradiol acetate, oestradiol benzoate, and the progestogens, for example progesterone, hydroxyprogesterone caproate, megestrol acetate, medroxaprogesterone acetate, chlormadinone acetate, cyproterone acetate, medrogestone, dydrogesterone, norethisterone, norethisterone acetate, norethisterone oenanthate, gestodene, levonorgestrel, etonogestrel, dienogest, danazol, norgestimat, lynestrenol, desogestrel and drospirenone.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2006 003 512.7, filed Jan. 24, 2006, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Film-shaped system capable of transmucosal buccal application and comprising
at least one steroid hormone from the group of the androgens, the progestogens and the oestrogens,
10-70% of a cyclodextrin or a cyclodextrin derivative, and
a film-forming agent disintegrating in an aqueous medium.

2. Film-shaped system according to claim 1, characterized in that
the androgen is testosterone, 7α-methyl-19-nortestosterone (MENT), MENT-17-acetate, 7α-methyl-11β-fluoro-19-nortestosterone (eF-MENT), eF-MENT-acetate, testosterone propionate, testosterone undecanoate, testosterone oenanthate, mesterolone, nandrolone decanoate, clostebol acetate, metenolone acetate,
the progestogen is progesterone, hydroxyprogesterone caprate, megestrol acetate, medroxaprogesterone acetate, chlormadinone acetate, cyproterone acetate, medrogestone, dydrogesterone, norethisterone, norethisterone acetate, norethisterone enanthate, gestodene, levonorgestrel, etonogestrel, dienogest, danazol, norgestimat, lynestrenol, desogestrel and drospirenone,
the oestrogen is 17-beta-oestradiol, ethinyloestradiol, oestradiol valerate, oestradiol cypionate, oestradiol acetate and oestradiol benzoate
or a mixture of the steroid hormones.

3. Film-shaped system according to claim 1, characterized in that the system contains fully dissolved 0.1-5% by weight, preferably 1-2% of the steroid hormones.

4. Film-shaped system according to claim 1, characterized in that the content of cyclodextrin or cyclodextrin derivative is 20-60% by weight.

5. Film-shaped system according to claim 4, characterized in that the content of cyclodextrin or cyclodextrin derivative is 40-60% by weight.

6. Film-shaped system according to claim 1, characterized in that the cyclodextrin is HP-beta cyclodextrin or the cyclodextrin derivative is sulfobutyl ether-beta cyclodextrin.

7. Film-shaped system according to claim 1, characterized in that the film-forming agent disintegrating in an aqueous medium is selected from the group comprising water-soluble polymers from the group of polyvinyl alcohols with a degree of hydrolysis of 75-99%, polyvinylpyrrolidone, polyethylene glycols, hydrophilic cellulose derivatives such as hydroxypropylcellulose, hydroxypropylmethylcellulose or carboxymethylcellulose, pullulan or maltose, hydrophilic starch derivatives such as carboxymethyl starch, alginates or gelatine, Eudragites, polyvinyl alcohols and celluloses.

8. Film-shaped system according to claim 1, characterized in that the content of organic solvents is equal to or less than 0.1% by weight, preferably equal to or less than 0.01% by weight.

9. Film-shaped system according to claim 1, characterized in that the preparation exhibits visible light transmission of equal to or greater than 50%.

10. Film-shaped system according to claim 1, characterized in that it contains 1-20% by weight of a plasticizer from the group of the mono- to trihydric alcohols with 2-4 carbon atoms, preferably 1,2-propylene glycol, polyethylene glycol or glycerol.

11. Film-shaped system according to claim 1, characterized in that the system contains 1-30% by weight, preferably 5-15% by weight of microcrystalline cellulose.

12. Film-shaped system according to claim 1, characterized in that the system contains an oestrogen and a progestogen as steroid hormones.

13. Film-shaped system according to claim 12, characterized in that the oestrogen is ethinyl-oestradiol.

14. Film-shaped system according to claim 12, characterized in that the oestrogen is 17beta-estradiol.

15. Film-shaped system according to claim 12, characterized in that the progestogen is gestodene.

16. Film-shaped system according to claim 12, characterized in that the progestogen is drospirenone.

17. Film-shaped system according to claim 15, characterized in that the oestrogen is ehinyl-oestradiol.

18. Film-shaped system according to claim 16, characterized in that the oestrogen is ethinyl-oestradiol.

19. Film-shaped system according to claim 16, characterized in that the oestrogen is 17beta-oestradiol.

20. Process for producing a film-shaped system capable of transmucosal buccal application, characterized in that
30-70% by weight of a cyclodextrine or of a cyclodextrine derivative, based on the final weight of the film-shaped system capable of transmucosal buccal application is dissolved in water
at least one steroid hormone from the group of the androgens, the progestogens and the oestrogens is added and subsequently
a water-soluble polymer (film-forming agent) is added,
if appropriate admixed with a plasticizer or microcrystalline cellulose,
spread out on a dehesive base material, preferably polyethylene (PE) or polyethylene terephthalate (PET) film,
dried and
singulated.

21. Use of a film-shaped system capable of transmucosal buccal application and consisting of steroid hormones from the group of the androgens, the progestogens and the oestrogens and 10-70% by weight of a cyclodextrin or a cyclodextrin derivative and a film-forming agent disintegrating in aqueous medium and if appropriate admixed with a plasticizer or microcrystalline cellulose for prophylaxis and therapy of androgen, progestogen and/or oestrogen deficiency diseases.
